(19) United States
(12) Patent Application Publication
(10) Pub. No.: US 2024/0321441 A1
(43) Pub. Date: Sep. 26, 2024

(54) MEDICAL DEVICE REGISTRATION MANAGEMENT

(71) Applicant: ICU Medical, Inc., San Clemente, CA (US)

(72) Inventors: Mark C. Rohlwing, Mesa, AZ (US); Rahul K R, Chennai (IN); Satyendra Singh Jadaun, Kota (IN); Rosaiah Allam, Chennai (IN); Hrishikesh Anil Dandekar, Pune (IN)

(21) Appl. No.: 18/187,485

(22) Filed: Mar. 21, 2023

(51) Int. Cl. G16H 40/60; G16H 20/17

(52) U.S. Cl. CPC G16H 40/60; G16H 20/17; A61M 5/142

(57) ABSTRACT

Techniques for managing the registration of medical devices with servers and/or other computing systems in an efficient manner are provided. These techniques may include use of partial or delayed registration. When a medical device, such as an infusion pump, initially connects to a server, such as a health management system or an intermediary, the medical device may register with the server. For a period of time after completion of the registration, the medical device may not be required to re-register when a network connection is interrupted and reestablished. To reestablish communications, a secure connection may be established using a process by which the medical device proves its identity. The medical device may then resume normal network communication using the secure connection, without performing a full registration process. Full registration may be delayed until a period of time has expired.

### CROSS-REFERENCE TO RELATED APPLICATIONS

All applications for which a foreign or domestic priority claim is identified in the Application Data Sheet as filed with the present application are hereby incorporated by reference and made part of this specification.

### TECHNICAL FIELD

This disclosure relates to the field of medical device management, and particularly to systems and methods for medical device communication.

### BACKGROUND

Electronic medical devices often have processors and other computing components. Such medical devices may execute software and communicate with other computing systems via a network. Secure network communication may involve the establishing secure connections based on the identities of the devices participating in the communication, and encryption and decryption of data transmitted via the secure connections.

### SUMMARY

Various techniques are described herein for managing the registration of medical devices with servers and/or other computing systems in an efficient manner that reduces the load on networks and servers processing registration requests. These techniques may include use of partial or delayed registration. When a medical device (e.g., an infusion pump) initially connects to a server (e.g., a medical device server or intermediary thereto), the medical device may register with the server. For a period of time after completion of the registration, the medical device may not be required to re-register when a network connection is interrupted and reestablished. To reestablish communications, a secure connection may be established using a process by which the medical device proves its identity. The medical device may then resume normal network communication using the secure connection, without performing a full registration process. Full registration may be delayed until a later time, so long as it is performed before a period of time has expired (e.g., the medical device's registration may expire every 24 hours). Thus, the registration load on networks and servers after network interruptions may be delayed or reduced altogether, which mitigates the risk that registration after an interruption will cause further interruptions. These and other embodiments are described in greater detail below with reference to FIGS. 1-5. Although many of the examples are described in the context of particular medical devices, functions, and hospital or clinical environments, the techniques described herein can be applied to other medical devices, functions, and hospital or clinical environments.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of medical devices registering with computing systems according to some embodiments.

FIG. 2 is a flow diagram of an illustrative routine for managing connections and registration of a medical device with other devices in a clinical network environment according to some embodiments.

FIG. 3 is a diagram illustrating data flows and interactions during a full registration process according to some embodiments.

FIG. 4 is a diagram illustrating data flows and interactions during a connection re-establishment process with delayed registration according to some embodiments.

FIG. 5 is a block diagram illustrating exchange of registration signals according to some embodiments.

### DETAILED DESCRIPTION OF ILLUSTRATIVE EMBODIMENTS

The present disclosure is directed to managing the registration of medical devices with servers and/or other computing systems in an efficient manner that reduces the load on networks and servers processing registration requests.

Some existing methods of communication between medical devices and servers or other computing systems in a clinical environment involve a full registration process whereby a medical device is required to register with a server in order to communicate with the server (e.g., to exchange clinical data, receive programming instructions, etc.). For example, a medical device may be required to provide logging data regarding events occurring on the medical device, manifest data regarding the current software and/or hardware configuration of the medical device, and the like. In this way, a server can ensure that the medical device has the proper components, is communicating using the proper protocols, etc. However, by requiring the exchange of a relatively large amount of data and performing processing of that data by a server to complete the registration, various networking issues may arise. If too many medical devices attempt to register with the server at the same time (e.g., after a network outage or server outage), the full registration processes can overwhelm the network and/or the server and, in extreme cases, can cause a condition similar to a distributed denial of service attack. If network signal availability is inconsistent in a wireless network environment, the repeated registration processes that occur on every reconnection to the network can consume bandwidth and further affect the network, potentially causing additional interruptions.

Some aspects of the present disclosure address the issues noted above, among others, through the use of partial or delayed registration, also referred to as “lazy registration,” instead of a full registration in certain cases. When a medical device, such as an infusion pump, initially connects to a server, such as a medical device server or an intermediary, the medical device may register with the server. For example, the medical device may perform a full registration in which the medical device provides logging data regarding events occurring on the medical device, manifest data regarding the current software and/or hardware configuration of the medical device, and the like. The server may process the registration data (e.g., to ensure that the medical device has the proper components, is using the proper communication protocols, etc.), and complete registration of the medical device. For a period of time after completion of the registration, the medical device may not be required to re-register when a network connection is interrupted and reestablished. During this time, the medical device and/or the server may in some embodiments send periodic signals (e.g., “heartbeats”) to indicate normal operation and continued connection. For example, the medical device may send heartbeat signals to the server (or vice versa). If the recipient of the heartbeat signals (e.g., the server in this example) does not receive a heartbeat signal for a period of time (e.g., equal to a predetermined number of heartbeat intervals), then the recipient can determine that the connection to the originator (e.g., the medical device in this example) has been lost.

To reestablish communications without the overhead of sending all registration related data after every interruption, a delayed registration process may be performed in which a secure connection may be established using a process by which the medical device proves its identity, such as by using the secure sockets layer (“SSL”) protocol or transport layer security (“TLS”) protocol with signed certificates. The medical device may then resume normal network communication using the secure connection, without performing a full registration process. The delayed registration is “lazy” in the sense that full registration may be delayed until a period of time has expired (e.g., the medical device's registration may expire every 24 hours). In some embodiments, full registration may be delayed regardless of how many times the medical device re-establishes communication with the server. In some embodiments, the medical device may be permitted to establish communication a limited number of times during a particular time period before a full registration is performed. In some embodiments, the full registration may be permitted or required before the next scheduled time (e.g., before 24 hours) if some critical information of the medical device has changed, such as metadata or device name.

Further aspects of the disclosure relate to methods and reasons for the medical device server to reject registration of a medical device. After a medical device registers with the medical device server-whether the most recent registration was a full registration, or a delayed registration in which a secure connection is established without a full registration—the medical device server may determine that it should no longer be connected to the medical device and may therefore reject the registration. The medical device server can perform one or more operations in such cases, including: disconnecting the network socket by which the medical device communicates with the medical device server; sending a registration rejection message to the medical device; revoking a security certificate of the medical device; other operations; or some combination thereof. For example, in the case of a cybersecurity event detected by the medical device server or about which the medical device server is otherwise notified, the medical device server can disconnect the network socket and revoke the security certificate of the medical device.

In some embodiments, medical devices may use a backoff algorithm when connecting to and/or registering with a server in order to avoid overloading the server when multiple medical devices are to connect and/or register. For example, if a medical device attempts to connect to and/or register with a server (whether an initial full registration, or a subsequent full registration after expiration or invalidation of the initial full registration), and the connection/registration is unsuccessful, the medical device may wait for a period of time before retrying. The period may be chosen using some degree of randomness (e.g., a value of either 0 or a predetermined slot time may be chosen using a pseudo random number generator). Upon failure of the retry attempt, the medical device may wait for a potentially longer period of time, chosen using some degree of randomness (e.g., a value of either 0 or a multiple of the slot value may be chosen using the pseudo random number generator). Subsequent retires may continue using exponentially larger ranges of possible wait times.

Additional aspects of the disclosure relate to types of messaging signals used during registration of a medical device, and the data represented by the messaging signals. Messages passed between medical devices, servers, and other computing devices in a clinical environment may be structured using signal definitions specifying the data required and/or permitted to be included in a message. In some embodiments, a top-level message signal definition may specify that certain fields are permitted to be included in a definition. Some of the fields may be relevant to registration of a medical device, such as a field for manifest data, a field for log data (e.g., log history), and a field for requesting registration and responding to the request. Some fields may take a single data item (e.g., one value from a range of possible values), while other fields may be more complex and take their own data structure. For example, the registration field may be defined using a separate registration signal definition that specifics the data required and/or permitted to be included, and the registration signal definition may include additional signal definitions in a nested manner, such as signal definition for registration request-specific data and a signal for registration response-specific data. By including both registration request-specific data and a registration response-specific data in a registration message, the entire registration process can be encapsulated in a single message that is ultimately received by the medical device and able to be processed in a stateless manner.

Various aspects of the disclosure will now be described with regard to certain examples and embodiments, which are intended to illustrate but not limit the disclosure. Although aspects of some embodiments described in the disclosure will focus, for the purpose of illustration, on particular examples of medical devices, registration protocols, message fields, and the like, the examples are illustrative only and are not intended to be limiting. In some embodiments, the systems and methods described herein may be applied to additional or alternative medical devices, registration protocols, message fields, etc.

### Overview of Example Clinical Environment

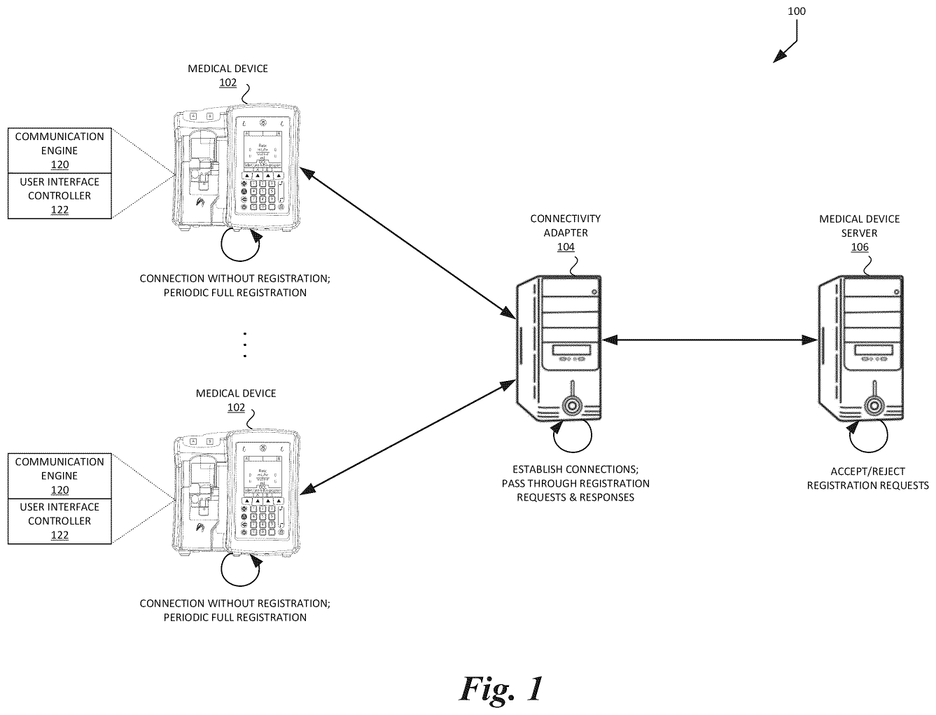

FIG. 1 shows a clinical environment 100 in which aspects of the present disclosure may be implemented for establishing network communications and registering medical devices. Devices of the clinical environment 100 may communicate with each other via one or more wired and/or wireless communication networks such as local area networks (“LANs”), virtual local area networks (“VLANs”), wide area networks (“WANs”), etc. The clinical environment 100 may include any number of medical devices 102 and other computing systems, such as a connectivity adapter 104 and a medical device server 106.

A medical device may 102 may be any electronic medical device, or medical device with an electronic component, configured to communicate with other devices over a communication network. In some embodiments, a medical device 102 may be an infusion pump. A medical device 102 may include various subsystems and/or other components. In some embodiments, as shown, the medical device 102 may include a user interface controller 122 (“UIC”) to control aspects of a user interface of the medical device 102, such as a display screen. The UIC 122 may be implemented using computer-readable memory and a computer processor, such as a system on a chip (“SOC”). The medical device 102 may also include a communication engine 120 (“CE”) to manage network communications to and/or from the medical device 102, to provide network security for the medical device 102 (e.g., a firewall), and the like. The CE 120 may be implemented using computer-readable memory and a computer processor, such as an SOC. In some embodiments, the CE 120 may be implemented using a physically separate computer processor that than the computer processor on which the UIC 122 is implemented. In this way, the CE 120 can serve as an intermediary between the UIC 122 and other systems and devices of the clinical environment 100.

In some embodiments, the medical device 102 may include additional and/or alternative components not shown. For example, if the medical device 102 is an infusion pump, the medical device 102 may include: a motor to administer medication from a medication container (e.g., a vial) to a patient; a motor controller to control the operation of the motor; a data store to store data and/or instructions used in operation of the medical device 102; various other components; or some combination thereof.

A connectivity adapter 104 may be a network component configured to communicate with other components of the clinical environment 100, such as medical devices 102 and the medical device server 106. In one embodiment, the connectivity adapter 104 servers as an intermediary between the medical devices 102 and the medical device server 106. For example, all messages communicated between medical devices 102 and the medical device server 106 may pass through the connectivity adapter 104. Thus, the connectivity adapter 104 can reduce load on the medical device server 106 by serving as an aggregation point for connections with medical devices 102, whereby separate connections from multiple medical devices 102 to the connectivity adapter 104 correspond to a single connection (or otherwise smaller number of connections) from the connectivity adapter 104 to the medical device server 106. In some cases, the connectivity adapter 104 is a network appliance with limited storage space (e.g., memory and/or persistent storage).

In some embodiments, one or more medical devices 102 may establish connections to the medical device server 106 without going through the connectivity adapter 104, or the connectivity adapter 104 may be excluded altogether.

The medical device server 106 may be a computing system configured to electronically communicate with other devices over a network. In some embodiments, the medical device server 106 may be implemented using one or more desktop computers, server computers, network appliances, or other computing systems that include one or more computer processors and network interfaces. The medical device server 106 may send data and/or instructions to a medical device 102 for use in performing medical procedures (e.g., administration of medication), receive data from a medical device 102 regarding a medical procedure (e.g., results of medication administration, alerts, etc.), process and store medical data, and the like. In some embodiments, the medical device server 106 may be part of, or may communicate with, one or more clinical IT systems. For example, the medical device server 106 may include a hospital information system (“HIS”) designed to manage medical, administrative, financial, and/or legal issues of a medical facility in which a medical device 102 is used. The HIS can include one or more electronic medical record (“EMR”) or electronic health record (“EHR”) systems.

In some embodiments, the clinical environment may include an HIS/EHR that is separate (not shown) from the medical device server 106. In some embodiments, one or more of the medical device server 106 and/or HIS/EHR may be implemented remotely from the clinical environment in which the medical devices 102 and connectivity adapter 104 are located. For example, the features and services provided by the medical device server 106, HIS/EHR, etc. may be implemented as web services consumable via one or more communication networks. In further embodiments, the medical device server 106, HIS/EHR, etc. are provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment

The example devices and systems of the clinical environment shown in FIG. 1 and described herein are illustrative only, and are not intended to be limiting, required, or exhaustive. In some embodiments, a clinical environment 100 may include additional, alternative, and/or fewer devices and/or systems. For example, although only two instances of a medical device 102 and one instance of a connectivity adapter 104 and medical device server 106 are shown in FIG. 1, in practice any number or combination of devices and systems may be included in a clinical environment 100. A single clinical environment 100 may have dozens, hundreds, or more individual medical devices 102, and the medical devices 102 may be the same as or different than each other.

### Medical Device Registration

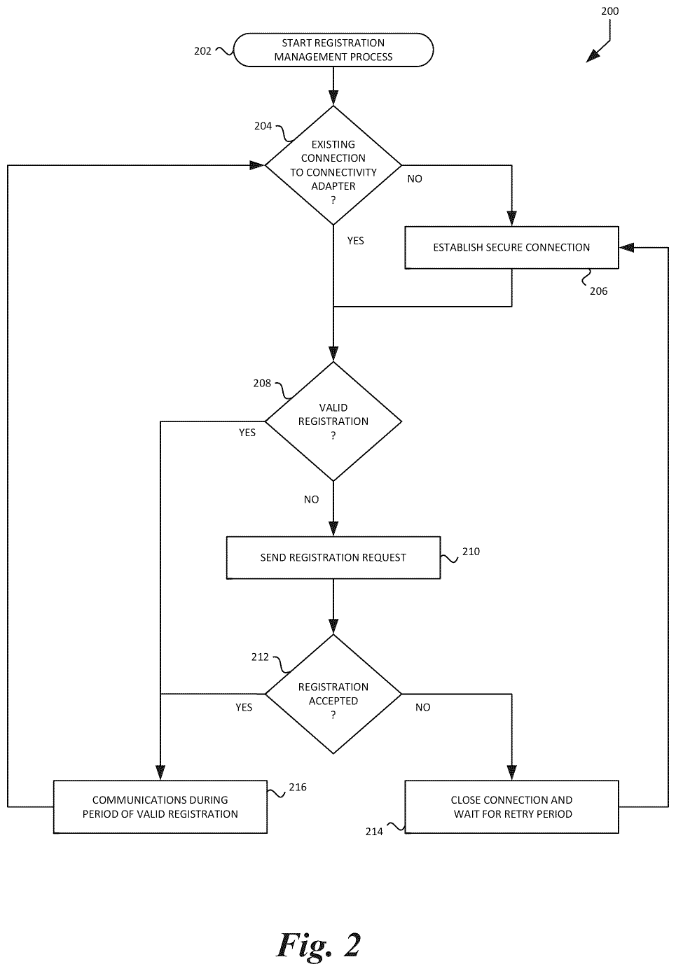

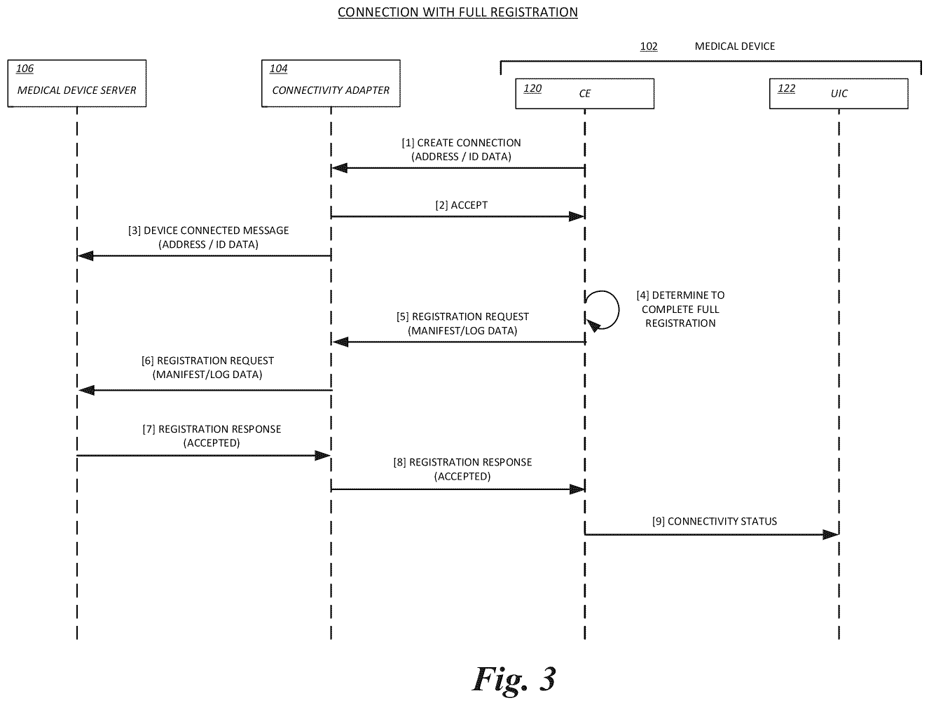

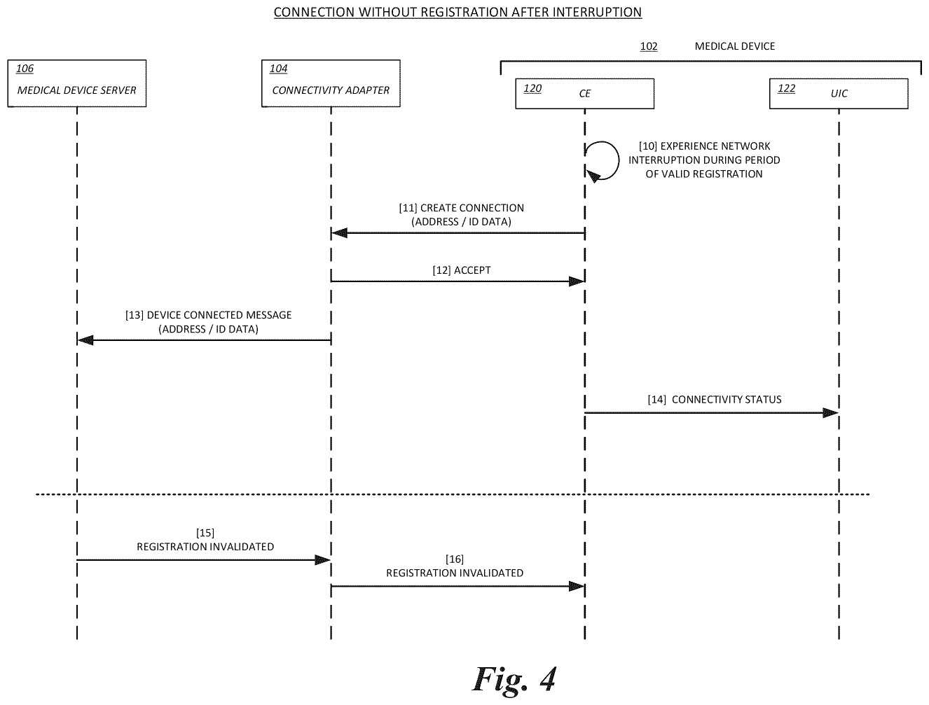

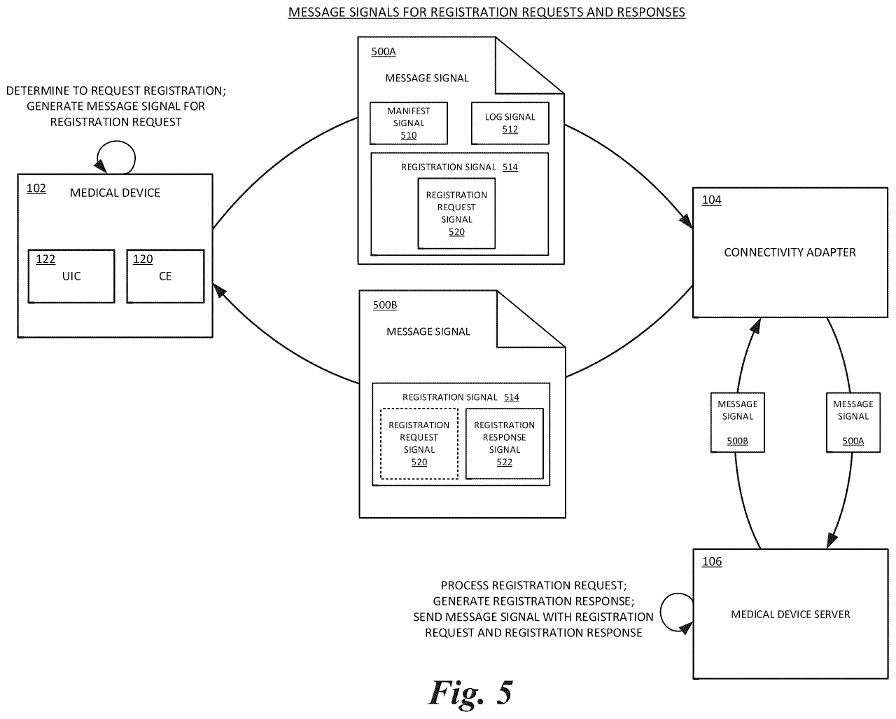

FIG. 2 is a flow diagram of an illustrative routine 200 for managing connections and registration of a medical device with other computing systems in a clinical network environment. Advantageously, a medical device 102 may use routine 200 to reconnect with a connectivity adapter 104 in case of a network interruption without requiring performance of a full registration process. The full registration process can be delayed until a later time to reduce the amount of network traffic and server processing required to reconnect after network interruptions. The routine 200 will be described with further reference to interactions and data flows shown in FIGS. 3 and 4, which illustrate a full registration and a delayed registration process, respectively. Additional reference will be made to the example messaging signals illustrated in FIG. 5.

In some embodiments, a medical device 102 may establish connections to the medical device server 106 without going through a connectivity adapter 104, and thus references to the connectivity adapter 104 in the description and illustration of the routine 200 may be replaced by the medical device server 106.

The routine 200 beings at block 202. The routine may begin in response to an event, such as powering on of a medical device 102. In some embodiments, the routine 200 may be performed by a CE 120 of a medical device 102.

At decision block 204, the CE 120 can determine whether there is an existing connection to the connectivity adapter 104. If not, the routine 200 may proceed to block 206. Otherwise, the routine 200 may proceed to decision block 208.

At block 206, CE 120 can establish a secure connection to the connectivity adapter 104. The CE 120 may initiate a secure connection protocol with the connectivity adapter 104, such as the SSL or TLS protocol, to establish the secure connection. Illustratively, the CE 120 may use a previously-obtained certificate to prove the identity of the medical device 102 to the connectivity adapter 104. Example routines and protocols for managing identity and secure communications among medical devices and other computing systems in a clinical network environment are described in PCT Patent Application No. PCT/US2022/076417, filed Sep. 14, 2022 and entitled Medical Device Communication Certificate Management, which is incorporated by reference herein and forms part of this specification.

At decision block 208, the CE 120 can determine whether a delayed registration for the connection is sufficient (e.g., a registration time period after a previously-completed full registration has not expired or the previously-completed full registration is otherwise still valid), or whether a full registration is required before further communications are permitted. If a delayed registration is sufficient, the routine 200 can proceed to block 216. Otherwise, if no full registration process has yet been completed or a full registration is otherwise required, then the routine 200 can proceed to block 210.

An example communication protocol for a medical device 102 to complete a full registration process is illustrated in FIG. 3. The full registration process may be completed when a medical device 102 has not previously been registered (e.g., when a medical device 102 is first deployed in a clinical environment) or when a prior valid registration has expired or become invalid. In some embodiments, registration may expire after a predetermined or dynamically determined period of time, referred to as a registration time period. For example, a medical device 102 may be required to complete a full registration process at least once every x hours (e.g., where x is a number such as 12, 24, 26, 48, etc.). In some embodiments, a registration may be invalidated in response to an event. For example, the software and/or hardware components of a medical device 102 may be changed, and the medical device 102 may no longer be permitted to communicate with other devices in a clinical environment until a full registration process is performed. As another example, a security event may occur, and the connectivity adapter 104 and/or medical device server 106 may invalidate the registration of one or more medical devices 102.

As shown in FIG. 3, at [1] the CE 120 may create a secure connection with the connectivity adapter 104. Illustratively, the CE 120 may open a web socket to the connectivity adapter 104 and may provide information, such as an address (e.g., an IP address), identity data (e.g., a secure certificate uniquely associated with the medical device 102), and the like. At [2] the connectivity adapter 104 may accept the connection request, and the connection may be established. In some embodiments, the connectivity adapter 104 may notify other systems of the secure connection with the medical device 102. For example, the connectivity adapter 104 may notify the medical device server 106 at [3] that the medical device 102 now has a connection with the connectivity adapter 104.

At [4] the CE 120 may determine to complete a full registration. For example, the CE 120 may determine at decision block 208 of routine 200 that no registration was previously completed, or that a previously valid registration is now expired or otherwise invalid. In response to this determination, the CE 120 may generate a registration request.

To generate a registration request, the CE 120 may obtain data regarding the software and/or hardware configuration of the medical device 102. The CE 120 may use the configuration data to generate a manifest of the hardware components of the medical device 102 (e.g., the types, versions, identifiers, etc. for individual components of the medical device 102), the software components of the medical device 102 (e.g., the types, versions, identifiers, etc. for individual applications and libraries of the medical device 102), and the like. For example, the CE 120 may generate a manifest that includes data regarding the version of the drug library currently installed on the medical device 102 to manage administration of medication. The CE 120 may also or alternatively obtain logging data regarding events (e.g., medication administration processes, alerts, manual operational overrides, etc.) occurring on the medical device 102. The CE 120 can include the manifest and/or logging data in a message to be sent to the connectivity adapter 104. An example of a registration message with manifest and/or logging data is shown in FIG. 5 and described in greater detail below.

Returning to FIG. 2, at block 210 the CE 120 can send a registration request message (in some cases with manifest and/or logging data) to the connectivity adapter 104 via the previously-established secure connection. Sending of the registration request message is also shown in FIG. 3 at [5]. The connectivity adapter 104 may process the request, or provide it to another system for processing. For example, the request may be forwarded to the medical device server 106 at [6].

The medical device server 106 may process the message with the registration request to determine whether to approve the registration. In some embodiments, the medical device server 106 may evaluate one or more registration criteria to determine whether to approve the registration. For example, the medical device server 106 may determine whether the medical device 102 is requesting to use a supported communication protocol, whether the medical device 102 is of a supported device class, whether a license is available for the medical device 102, whether the name or other identifier of the medical device 102 is valid and not currently in use for another registration, whether the medical device 102 satisfies some other criterion, or some combination thereof. In some embodiments, the medical device server 106 may evaluate manifest and/or logging data associated with the registration request. The evaluation may be performed to determine whether the hardware and/or software configuration of the medical device 102 has changed or should be changed. For example, the medical device server 106 may determine whether the medical device 102 has the appropriate drug library and, if not, require an update to the drug library in order to approve the registration, or provide an updated drug library in connection with approving the registration request.

The medical device server 106 may generate a message with a registration response. The message may indicate whether the registration has been approved, a reason for rejecting the registration (if any), and/or other related registration information. An example message with a registration response is shown in FIG. 5 and described in greater detail below. The medical device server 106 may provide the message to CE 120 at [7], such as through the intermediary connectivity adapter 104 which can forward the message at [8]. Once the medical device 102 has completed a registration process, subsequent connections to the connectivity adapter 104 may be established without re-registering so long as the previous registration is still valued, as shown in FIG. 4 and described in greater detail below.

Returning to FIG. 2, at decision block 212, the CE 120 can determine whether registration has been accepted. If so, the routine 200 may proceed to block 216, where the medical device 102 can send and receive communications via the secure connection while the registration is valid (e.g., during a period of valid registration). In some embodiments, the CE 120 may provide an indication of connectivity status (e.g., connectivity status information) to one or more other components of the medical device 102, such as to the UIC 122, as shown in FIG. 3 at [9].

If the CE 120 determines at decision block 212 that the registration request has not been accepted, the routine 200 may proceed to block 214 where the CE 120 can retry a registration request. In some embodiments, the CE 120 also closes the secure connection prior to retrying the registration request.

In some embodiments, the CE 120 may wait for a retry period of time to pass before retrying the registration process. In this way the CE 120 can avoid repeated retries in cases where the connectivity adapter 104, medical device server 106, network, etc. are experiencing a high volume of requests or are otherwise unable to respond to registration requests in a timely manner. In some embodiments, the CE 120 may enact different registration retry logic depending upon the reason the registration was rejected. For example, the medical device server 106 may return a rejection code indicating the registration of the medical device 102 is rejected because the medical device 102 is not licensed. In response to such a rejection code, the CE 120 may try to reconnect after a period of time (e.g., in x hours). As another example, if the rejection code returned by the medical device server 106 indicates that the medical device server 106 is not ready or available to complete the registration, the CE 120 may try to reconnect after a shorter period of time (e.g., in y minutes).

FIG. 4 illustrates a continuation of the operations shown in FIG. 3, in which the medical device 102 has already completed the registration process. Moreover, the registration remains valid. For example, after the successful registration described above there may be an x-hour period of time (where x is a number such as 12, 24, 36, 48, etc.) during which the medical device 102 is not required to complete another full registration, even when reconnecting to the connectivity adapter 104. The medical device 102 may therefore continue to operate and send/receive network communications (e.g., communicate to/from the connectivity adapter 104 at block 216).

At the medical device 102 may experience a network interruption, such as a wireless network disconnection, a restart of the medical device 102, a restart of the connectivity adapter 104, or the like. The CE 120 may determine (e.g., at decision block 204 of routine 200) that there is no longer a connection to the connectivity adapter 104. In response, the CE 120 may begin re-establishing a secure connection (e.g., at block 206 of routine 200) with the connectivity adapter 104 by creating a connection to the connectivity adapter 104 at [11]. The connection may be created as described in greater detail above with respect to creation of the prior connection to the connectivity adapter 104 at [1].

Illustratively, the CE 120 may open a web socket to the connectivity adapter 104 and may provide information, such as an address (e.g., an IP address), identity data (e.g., a secure certificate uniquely associated with the medical device 102), and the like. At the connectivity adapter 104 may accept the connection request, and the connection may be established. In some embodiments, the connectivity adapter 104 may notify other systems of the secure connection with the medical device 102. For example, the connectivity adapter 104 may notify the medical device server 106 at that the medical device 102 now has a connection with the connectivity adapter 104. In some embodiments, the CE 120 may notify other components of the medical device 102, such as the UIC 122, of the current connectivity status at [14].

Because the established connection is secure and the medical device 102 has proved its identity as part of the process of establishing the secure connection, the CE 120 may be permitted to communicate with the connectivity adapter 104 (and other systems such as the medical device server 106) as long as a registration process was previously completed and is still valid. Thus, at decision block 208 in this example, routine 200 may proceed to block 216. In some embodiments, the medical device 102 can determine when to complete a re-registration (e.g., based on predetermined waiting periods, expirations etc.). In some embodiments, the connectivity adapter 104 or medical device server 106 may determine when re-registration should be performed, and may send a message to the medical device 102 to request registration, or may invalidate the registration so that the next time the medical device 102 attempts to the communicate with the connectivity adapter 104, the medical device 102 will be notified that the registration has become invalid.

In some embodiments, after a medical device 102 registers with the medical device server 106, the medical device server 106 (and/or the connectivity adapter 104) may determine that it should no longer be connected to the medical device 102 and may therefore invalidate the registration. As shown in FIG. 4 at [15], the medical device server 106 may send a message regarding invalidation of the registration, and the connectivity adapter 104 may pass the message to the medical device at [16]. For example, the medical device server 106 may determine that a cybersecurity event has occurred, that the registration has expired, or that some other event has occurred to invalidate the registration.

### Example Registration Messaging

FIG. 5 is a block diagram of a medical device 102 exchanging messages for registration with a connectivity adapter 104. The CE 120 may determine to initiate a full registration process by requesting registration. In some embodiments, the determination may be made at decision block 208 of routine 200. For example, the CE 120 may determine that a prior registration has expired or been invalidated, or that there has been no prior registration.

The CE 120 may generate a message signal 500A to request registration of the medical device 102. The message signal 500A (also referred to simply as a “message”) may be structured according to a signal definition. In some embodiments, a signal definition may specify any number of fields that are required and/or permitted to be included in a message. Individual fields may be identified using labels and/or identification numbers. In addition, individual fields may be defined in terms of the type and/or structure of data to be included in the fields. For example, a field may be defined for data of a particular data type, such as a string, integer, floating point number, etc. As another example, a field may be defined for an enumeration, such as a set of possible numeric values mapped in a one-to-one manner to a set of possible alphanumeric tokens. As a further example, a field may be defined for a separate data structure, such as another signal definition that may itself include any number fields, field data types or structures, etc. In this way, signal data structures may be nested multiple levels deep, re-used across different top-level signals, etc.

In the example shown in FIG. 5, the message signal 500A may include a field for a manifest signal 510, a field for a log signal 512, and a field for a registration signal 514. The various signals may be defined by their own signal definitions, and may include any number of fields, field types, etc. For example, as shown, the registration signal 514 may include a field for a registration request signal 520. The registration request signal 520 may be defined by its own signal definition, and may include any number of fields, field types, etc.

To generate message 500A, the CE 120 may obtain data regarding the software and/or hardware configuration of the medical device 102. The CE 120 may use the configuration information to generate a manifest signal 510 representing the hardware components of the medical device 102 (e.g., the types, versions, identifiers, etc. for individual components of the medical device 102), the software components of the medical device 102 (e.g., the types, versions, identifiers, etc. for individual applications and libraries of the medical device 102), and the like. For example, the CE 120 may generate a manifest signal 510 that includes data regarding the version of the drug library currently installed on the medical device 102 to manage administration of medication.

The CE 120 may also or alternatively obtain logging data regarding events (e.g., medication administration processes, alerts, manual operational overrides, etc.) occurring on the medical device 102. The CE 120 may use the logging data to generate a log signal 512.

To make a registration request, the CE 120 may generate a registration signal 514 with a registration request signal 520. The CE may then package the data from all of the signals into message 500A, and send message 500A to the connectivity adapter 104.

The connectivity adapter 104 may pass the message 500A to some other system for processing, such as the medical device server 106. The medical device server 106 may evaluate the message 500A and determine whether to approve the registration. The determination may be based on any number of factors. In some embodiments, the medical device server 106 may evaluate the content of the message 500A to determine whether to approve registration of the medical device 102. For example, the medical device server 106 may determine whether the requested communication protocol is supported, whether the device class of the medical device 102 is supported, whether there are licensing restrictions that prevent registration, whether the name or other identify of the medical device 102 is unknown or an inappropriate duplicate, etc. As another example, the medical device server 106 may evaluate data in the manifest signal 510 to determine whether the current hardware and/or software configuration of the medical device 102 is to be updated prior to, or in connection with, registration of the medical device 102. In some embodiments, the medical device server 106 may make a registration acceptance determination based partly or entirely on factors outside of the message 500A. For example, the medical device server 106 may determine that it does not have the capacity to fully process the registration at the present time, and may therefore reject the registration request.

The medical device server 106 may generate a message 500B in response to the request. The message 500B may include a registration response signal 522 that may or may not be part of a registration signal. In some embodiments, as shown, the message 500B may also include some or all of the data from the message 500A that was included the registration request, such as registration request signal 520.

The registration response signal 522 may be defined by its own signal definition, and may include any number of fields, field types, etc. For example, the registration response signal 522 may include a value that corresponds to an enumeration in which one enumerated item indicates approval, and one or more other enumerated items indicate rejection and/or a reason for rejecting the registration request. Upon receipt of the message 500B, the medical device 102 can determine whether the registration was approved and, if no, a reason why registration was not approved.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a computer processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A computer processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A system for managing communications for medical devices, the system comprising:
a medical device comprising a communication engine and a user interface controller; and
a connectivity adapter that serves as an intermediary between the medical device and a medical device server;
wherein the medical device is configured to:
establish a first secure connection with the connectivity adapter based at least partly on a signed certificate uniquely associated with the medical device;
send, to the connectivity adapter via the first secure connection, a request for registration of the medical device with the medical device server;
receive, from the connectivity adapter via the first secure connection, a response representing an approval of the registration; and
subsequent to approval of the registration:
exchange, via the first secure connection, a first plurality of network communications with the medical device server;
determine that the first secure connection has been disconnected;
establish a second secure connection with the connectivity adapter based at least partly on the signed certificate;
determine that a time period, after which a second request for registration of the medical device is to be sent, has not expired; and
exchange, via the second secure connection, a second plurality of network communications with the medical device server.

2. The system of claim 1, wherein the connectivity adapter is configured to:
forward the request for registration to the medical device server;
receive the response from the medical device server; and
forward the response to the medical device.

3. The system of claim 1, wherein the medical device is further configured to:
determine, subsequent to exchanging the second plurality of network communications, that the registration is invalid;
establish a third secure connection with the connectivity adapter based at least partly on the signed certificate; and
send, to the connectivity adapter via the third secure connection, a third request for registration of the medical device with the medical device server.

4. The system of claim 1, wherein the medical device is further configured to:
determine, subsequent to exchanging the second plurality of network communications, that the registration is invalid; and
send, to the connectivity adapter via the second secure connection, a third request for registration of the medical device with the medical device server.

5. The system of claim 4, wherein to determine that the registration is invalid, the medical device is configured to determine that a registration time period has expired.

6. The system of claim 4, wherein to determine that the registration is invalid, the medical device is configured to determine the medical device server has invalidated the registration.

7. The system of claim 4, wherein the medical device is further configured to:
receive, from the connectivity adapter via the second secure connection, a second response representing a registration rejection; and
determine, in response to the registration rejection, to wait for a predetermined time period before sending a third request for registration.

8. The system of claim 1, wherein the medical device is further configured to:
generate a log signal comprising data representing a log of events associated with the medical device;
generate a manifest signal comprising data representing a manifest of hardware and software components of the medical device; and
generate the request for registration comprising the log signal and the manifest signal.

9. The system of claim 1, wherein the medical device comprises an infusion pump.

10. The system of claim 1, further comprising the medical device server, wherein the medical device server comprises one or more computing devices configured to:
evaluate the request with respect to one or more registration criteria;
determine, based on results of evaluating the request, to approve the registration; and
generate the response.

11. An infusion pump comprising:
a first processor configured to:
determine that a first network connection to a server has been interrupted;
determine that a registration time period has not passed, wherein a first registration of the infusion pump with the server expires after the registration time period has expired;
establish a second network connection with the server;
exchange a first plurality of communications with the server via the second network connection;
subsequent to exchanging the first plurality of communications with the server:
determine that a second registration of the infusion pump with the server is to be obtained;
establish a third network connection with the server;
complete a registration protocol to obtain the second registration; and
exchange, via the third network connection, a second plurality of network communications with the server;
a display configured to display information regarding at least one of a patient or a medication; and
a motor controller configured to cause the medication to be administered to the patient from a medication container.

12. The infusion pump of claim 11, further comprising a second processor configured to control the display, wherein the first processor is further configured to send connectivity status information to the second processor in response to completing the registration protocol.

13. The infusion pump of claim 11, wherein the first processor is further configured to send connectivity status information to the motor controller in response to completing the registration protocol.

14. The infusion pump of claim 11, wherein to determine that the second registration is to be obtained, the first processor is configured to determine that the registration time period has expired.

15. The infusion pump of claim 11, wherein to determine that the second registration is to be obtained, the first processor is configured to determine the server has invalidated a previously-completed registration.

16. The infusion pump of claim 11, wherein the first processor is further configured to:
generate a log signal comprising data representing a log of events associated with the infusion pump;
generate a manifest signal comprising data representing a manifest of hardware and software components of the infusion pump; and
generate a registration request comprising the log signal and the manifest signal.

17. A medical device is configured to:
establish a first secure connection with a medical device server based at least partly on a signed certificate uniquely associated with the medical device;
send, to the medical device server via the first secure connection, a request for full registration of the medical device with the medical device server;
receive, from the medical device server via the first secure connection, a response representing an approval of the full registration; and
subsequent to approval of the full registration:
determine that the first secure connection has been disconnected;
determine that a time period, after which a second request for full registration of the medical device is to be sent, has not expired; and
execute a lazy registration of the medical device with the medical device server, wherein the lazy registration comprises:
establishment of a second secure connection with the medical device server based at least partly on the signed certificate; and
delay of the second request for full registration of the medical device with the medical device server.

18. The medical device of claim 17, wherein the request for full registration comprises a manifest signal and a log signal, wherein the manifest signal represents one or more hardware components of the medical device and one or more software components of the medical device, and wherein the log signal represents one or more events that have occurred on the medical device.

19. The medical device of claim 17, wherein to execute the lazy registration, the medical device is further configured to delay the second request for full registration until a period of time associated with the full registration has expired.

20. The medical device of claim 17, further configured to send, to the medical device server, the second request for full registration based on the time period being expired.
